(19) United States
(12) Patent Application Publication
(10) Pub. No.: US 2004/0219514 A1
(43) Pub. Date: Nov. 4, 2004

(54) Caml-binding peptides

(76) Inventors: Alexandru R. Moise, Seattle, WA (US); Wilfred A Jeffries, Vancouver (CA); Timothy Z Vitalis, Vancouver (CA); Jason R Grant, Vancouver (CA)

Correspondence Address: BOZICEVIC, FIELD & FRANCIS LLP, 200 MIDDLEFIELD RD, SUITE 200, MENLO PARK, CA, 94025, US

(21) Appl. No.: 10/433,919

(22) PCT Filed: Dec. 7, 2001
(86) PCT No.: PCT/CA01/01769

(30) Foreign Application Priority Data
Dec. 7, 2000 (CA) 2 325610
Mar. 2, 2001 (CA) 2 335411

(51) Int. Cl. C12Q 1/70; C12N 7/00; C07K 14/075

(52) U.S. Cl. 435/5; 435/235.1; 530/324

(57) ABSTRACT

This invention provides peptides that bind to CAML, and nucleic acids encoding such peptides. These peptides and nucleic acids are useful for targeting CAML and for modulating of CAML activity so as to affect immune or inflammatory response or to evade apoptosis. Also provided are vectors for expressing such peptides and implantable medical devices comprising the peptides or nucleic acids.

### FIELD OF THE INVENTION

This application relates to modulation of apoptosis and immune response.

### BACKGROUND OF THE INVENTION

Viral infection is a cellular injury, and can result in the induction of programmed cell death (apoptosis) of the host cell. Many viruses, particularly persistent DNA viruses modify the apoptotic response of a cell to allow continued virus replication. Apoptosis can be induced by the members of the TNF receptor super-family such as Fas (APO-1 or CD95) and p55 Tumor Necrosis Factor Receptor (p55 TNFR) as well as the death domain-containing receptors 3, 4 and 5 (DR3, DR4 and DR5, respectively). The intracellular factors responsible for death of the cell are highly conserved across species and are the target of viral inhibitors of apoptosis.

It appears that proteins belonging to very different classes of virus have evolved to block the same cellular apoptotic event. This convergent evolution is evidenced by the classification of viral inhibitors of apoptosis. For example, adenovirus E1B 55K, SV40 Large T antigen and human papilloma virus E6 inhibit p53-mediated lysis. The cellular survival factor Bcl-2 is mimicked by adenovirus E1B 19K, Epstein-Barr virus BHRF1 and African swine fever virus LMW5-HL. Members of the Interleukin 1b Converting Enzymes (ICE)-family of terminal proteolytic enzymes, also known as caspases, are blocked by baculovirus p35 and crmA, the cowpox serpin protein. The adenovirus E3/10.4K and E3/14.5K proteins downregulate surface Fas, while the Inhibitors of Apoptosis (IAP) family of baculovirus and mammalian homologues interact with the TNF-α receptor associated factors (TRAFs) therefore blocking the signalling cascade that leads to the recruitment of caspases. The activation of FADD-like interleukin-1beta-converting enzyme (FLICE), also known as caspase-8, through Fas is blocked by viral-FLICE-inhibitory proteins (vFLIPs), found in the genomes of various types of herpesvirus and by the E3/14.7K of adenovirus.

Adenovirus (Ad) is a very common human pathogen that results in persistent infections of the respiratory or gastrointestinal tract. Persistent infections stem from an elaborate evasion of the host defense mechanisms. The adenovirus genes responsible for immune evasion map to the Early 3 (E3) region of the Ad genome. The persistence, ease of infection and weak pathogenesis have made adenovirus suitable as vectors for gene therapy. Currently, Ad gene transfer vectors are the most efficient technique available for in vivo gene transduction. In the case of Ad vectors the genetic makeup of the original vectors was designed to accommodate large fragments of DNA for the transduced gene, to the expense of areas of the adenoviral genome that were considered dispensable. The E3 region was one of the first areas to be replaced.

The 6.7K protein encoded by the E3 region (E3/6.7K) does not have any significant homology to any other known proteins. It is well conserved between group C Ad2 and Ad5 adenovirus and between group B Ad3, Ad7 and Ad35 adenovirus. The Ad2 E3/6.7K protein (Wilson-Rawls et al., (1990) Virology 178:204-212) has been shown to be an integral membrane protein localized to the endoplasmic reticulum (ER) (Wilson-Rawls and Wold, (1993) Virology 195:6-15). The protein is present in two forms, one unglycosylated with an apparent molecular weight of 8 kDa and one glycosylated with an apparent weight of 14 kDa. The protein, though targeted to the ER, does not have a cleavable signal sequence, but it has a hydrophobic central region that could act as a signal anchor (Wilson-Rawls et al.,(1994) Virology 201:66-76).

The major impediment for the success of Ad vectors as well as all the other gene transfer technologies is the unexpectedly strong immune response to cells infected by a modified Adenovirus. The strong immune response to modified Ad vectors appears to be mediated by the circulating cytokine Tumor Necrosis Factor (TNF)α and by the innate immune response. The negative effects of an immune response might be alleviated by implementing immunomodulatory proteins that allow the vector and the transduced cells to survive the immune response.

The evasion of immune response is also a central impediment to the establishment of successful transplant technology as well as the treatment of autoimmune and neurodegenerative diseases. Apoptosis of the affected organ is often the result of neurodegenerative inflammatory disease. Factors that prevent apoptosis could lead to better therapies for these conditions.

Cell culture reactor expression systems are typically limited only by the ability of cells to grow and produce proteins of interest. As cells grow, they reach densities where protein production stops and producer cells undergo apoptosis in response to factors that are currently poorly characterized. There is potential for improving protein yield by avoiding the apoptotic response of cells grown in culture by including an antiapoptotic protein in the makeup of the cell.

CAML (also known as “calcium-signal modulating cyclophilin ligand”; U.S. Pat. Nos. 5,523,227 and 5,969,102) is located in cytoplasmic vesicles and regulates Ca²⁺ influx by modulating intracellular Ca²⁺ release. CAML affects calcineurin in response to an extrinsic signal and has been shown to be important in lymphocyte activation. CAML is involved in activation of transcription factors in lymphocytes, including T-cell transcription factor (NF-AT) and is important in modulation of immune response or modulation of apoptosis.

A lymphocyte surface receptor that binds CAML has been discovered and the human and murine sequences have been published. Thins protein is termed “transmembrane activator and CAML interactor” (TACI) and has been the focus of much attention in view of the interaction of TACI with CAML and its role in lymphocyte activation, particularly in B-cells (see: U.S. Pat. No. 5,969,102; Von Bulow, G. & Bram, R. J. (1997) Science, 278:138-141; Von Bulow, G., et al. (2000), Mammalian Genome, 11:628-632; Xia, X., et al. (2000), J. Exp. Med., 192:137-143; and, Ware, C. F. (2000), J. Exp. Med., 192:F35-F37). TACI is reported to have a N-terminal extracellular domain, a centrally located transmembrane domain, and a C-terminal intracellular domain. Investigation of sequence homology with respect to TACI only revealed homology between portions of the N-terminal domain of TACI (representing a TNFR_NGFR repeat motif in the regions of amino acids 33-104 of human TACI),and a number of growth factor receptors (U.S. Pat. No. 5,969,102). In the 293 amino acid human sequence of TACI, the transmembrane domain is thought to reside at about amino acids 167-186. First reports with respect to binding of TACI to CAML indicated that the CAML-binding region is in the C-terminus domain of TACI, since CAML-binding did not occur with amino acids 1-168 of human TACI (U.S. Pat. No. 5,969,102; and, Von Bulow, G. & Bram, R. J. (1997) [supra]). Results published more recently seem to show that amino acids 1-212 or 1-233 of human TACI are capable of binding to CAML (Xia, X. Z., et al. [supra]).

### SUMMARY OF THE INVENTION

It has now been discovered that adenovirus E3/6.7L prevents inflammation, apoptosis and the cellular damage response following viral infection. The presence of the E3/6.7K protein correlates with reduced inflammatory response in the lungs of virally infected mice. Transfected cells that express the E3/6.7K protein are now shown to be protected against apoptosis induced by TNF-α. TNF-α induced release of arachidonic acid is significantly reduced in cells expressing transfected E3/6.7K. Efflux of calcium ions from the ER is also reduced in the presence of E3/6.7K. Therefore, the mechanism of action of E3/6.7K does involve maintenance of calcium ion homeostasis. The release of calcium ions from the ER is known to be important for the generation of mediators of inflammation, apoptosis and of the cellular damage response.

E3/6.7K has no sequence homology to any of the previously described inhibitors of apoptosis. E3/6.7K therefore represents a member of a new class of modulators of apoptosis particularly in lymphocytes) and the immune response, and which are useful as modulators of inflammation. This new class of modulators act through alteration of Ca²⁺ influx and thus may be inhibiting or promoting in their effects.

By this invention, it is now known that E3/6.7K binds to CAML and inhibits Ca²⁺ influx, thus resulting in inhibition of apoptosis and inflammation. Binding by E3/6.7K fragments to full length CAML has been determined by immunoprecipitation and by yeast two-hybrid assays.

Examples of portions of E3/6.7K capable of interacting with CAML are a 26 amino acid domain situated at amino acids 32 to 57 of Ad2 and amino acids 34 to 59 of Ads, and in a corresponding 26 amino acid tract of Ad3. Such E3/6.7K domains are collectively termed herein “6.7-effector domain” (SED). Surprisingly, part of this domain shares homology with the putative transmembrane domain of TACI; contrary to the previous published reports with respect to TACI binding to CAML. Examples of the CAML-binding domains in TACI which correspond to SED, include domains of about 28 amino acids in length found from about amino acids 163-189 of human TACI and from about amino acids 126-152 or 153 of mouse TACI.

With knowledge of the CAML-binding domain of TACI and E3/6.7K, a CAML-binding motif has been determined and has been employed to identify known proteins which share this motif, thereby making such proteins available for use in CAML-binding.

One aspect of the invention involves the provision of and use of CAML-binding motifs that may be used to confer CAML-binding function on a ligand or other moiety intended to bind to CAML. Such motifs may in themselves modulate CAML function or may be used to facilitate CAML modulation by permitting binding of a CAML modulating ligand or moiety to CAML. The motifs may also be used to permit binding of moieties which are not intended to modulate CAML function (such as a CAML specific labeling moieties).

In alternative embodiments, the CAML-binding motifs may comprise sequences of 26, 27 or 28 amino acids, such as follows:

| | | |
|---|---|---|
| LALLCLRVAACCTHVCTYCQ | (from Ad2 E3/6.7K) | SEQ ID NO:1 |
| LFKRWG; | | |
| LTLLCLRLAACCVHICIYCQ | (from Ad5 E3/6.7K) | SEQ ID NO:2 |
| LFKRWG; | | |
| VLILCYLYTPCCAYLVILCC | (from Ad3 E3/1.6K) | SEQ ID NO:3 |
| WFKKWG; | | |
| VYSTLGLCLCAVLCCFLVAV | (from human TACI) | SEQ ID NO:4 |
| ACFLKKR; | | |
| LYCTLGVCLCAIFCCFLVAL | (from murine TACI) | SEQ ID NO:5 |
| ASFLRRRG. | | |

In alternative embodiments, the CAML-binding motif of this invention may comprise a sequence having any one of the following consensus sequences, wherein: a “x” represents any amino acid with the number of amino acids (or range of possible numbers of amino acids) being indicated by a bracketed number or numbers following “x”; and, single letter amino acid abbreviations within square brackets represent alternative amino acids at a single position:

(a) C-C-x(2)-[FILV]-[ACV]-x(2)-[CS]-x(3)-[KR]-[KR];

(b) C-C-x(2)-[ILV]-[ACV]-x(2)-[CS]-x(3)-[KR]-[KR];

(c) C-x(3,5)-C-C-x(2)-[FILV]-[ACV]-x(2)-[CS]-x(3)-[KR]-[KR];

(d) L-x(2,3)-C-x(4,5)-C-C-x(2)-[ILV]-[ACV]-x(2)-[CS]-x(3)-[KR]-[KR]; and,

(e) L-x(1,2)-Cx(5)-C-C-x(2)-[ILV][ACV]-x(2)-[CS]-x(3)-(KR]-[KR].

In alternative embodiments, the CAML-binding motifs of the invention may be derived from or used as part of known proteins or fragments of known proteins, including those set out in the following list in which the accession number of the database reference is followed by the name and source of the protein, together with identification of the amino acid sequence and its position within the protein corresponding to consensus sequence (b) above.

[1] SWISS-PROT: E316_ADE03 P11320

Early E3 16 KDA glycoprotein

Human adenovirus type 3

129-142 CCAYLVILCCWFKK; SEQ ID NO:6

[2] SWISS-PROT: SY61_DISOM P24505

Synaptotagmin A (synaptic vesicle protein O-P65-A)

Discopyge ommata (Electric ray)

79-92 CCFCICKKCLLKKK; SEQ ID NO:7

[3] SWISS-PROT: SY62_DISOM P24506

Synaptotagmin B (synaptic vesicle protein O-P65-B)

Discopyge ommata (Electric ray)

96-109 C CLCICKKCCCKKK; SEQ ID NO:8

[4] SWISS-PROT: SYT1_BOVIN P48018

Synaptotagmin I (P65)

Bos taurus (bovine)

75-88 CCFCICKKCLFKKK; SEQ ID NO:9

[5] SWISS-PROT: SYT1_CHICK P47191

Synaptotagmin I (P65)

Gallus gallus (chicken)

77-90 CCFCLCKKCLFKKK; SEQ ID NO:10

[6] SWISS-PROT: SYT1_HUMAN P21579

Synaptotagmin I (P65)

Homo sapiens (human)

75-88 CCFCICKKCLFKKK; SEQ ID NO:11

[7] SWISS-PROT: SYT1_MOUSE P46096

Synaptotagmin I (P65)

Mus musculus (mouse)

74-87 CCFCVCKKCLFKKK; SEQ ID NO:12

[8] SWISS-PROT: SYT1_RAT P21707

Synaptotagmin I (P65)

Rattus norvegicus (rat)

74-87 CCFCVCKKCLFKKK; SEQ ID NO:13

[9] SWISS-PROT: SYT2_MOUSE P46097

Synaptotagmin II

Mus musculus (mouse)

82-95 CCFCICKKCCCKKK; SEQ ID NO:14

[10] SWISS-PROT: SYT2_RAT P29101

Synaptotagmin II

Rattus norvegicus (rat)

82-95 CCFCICKKCCCKKK; SEQ ID NO:15

[11] TrEMBL: 014836 014836

Transmembrane activator and CAML interactor

Homo sapiens (human)

176-189 CCFLVAVACFLKKR; SEQ ID NO:16 (or, amino acids 139-152 of murine TACI: CCFLVALASFLRRR; SEQ ID NO:17)

[12] TrEMBL: Q64830 Q64830

URF E3A 7.1K

Human adenovirus type 5

44-57 CCVHICIYCQLFKR; SEQ ID NO:18

[13] TrEMBL: Q9Q8F7 Q9Q8F7

M147R

Myxoma virus

126-139 CCTGLASVCKYTKK; SEQ :) NO:19

Peptides of this invention may comprise an amino acid sequence corresponding to he above-described CAML-binding motifs combined with additional amino acids selected in order for the peptide to affect CAML function.

Peptides of this invention will have a minimum of about 14 amino acids. While no specific maximum length of peptides (including proteins comprising peptides) of this invention is contemplated and may comprise (for example) up to about 300 amino acids, peptides of this invention selected or employed for purposes of CAML-binding will typically have a maximum length of about 100, preferably less than 100 amino acids. Preferably, peptides of this invention will have a number of amino acids selected from a number from 14-60, more preferably from 18-60, more preferably from 20-60, more preferably from 25-60, more preferably from 25-50. For example, peptides of this invention may consist of from about 20 to about 75 amino acids; or, from about 25 to about 60 amino acids; or, from about 26 to about 40 amino acids; or, from about 26 to about 35 amino acids; or, from about 26 to about 30 amino acids. Any combination of these examples of minimum and maximum lengths may be employed.

This invention does not include the use of previously identified, native (e.g. full length) human and murine TACI proteins or known fragments of such proteins for CAML-binding, CAML modulation, or modulation of apoptosis or immune response. However, this invention does include peptides and the use of peptides derived from TACI as described herein Such TACI derived proteins and fragments may comprise or consist of the 27 and 28 amino acid CAML binding peptides from human and murine TACI described above and the CAML-binding motifs comprising amino acids 176-189 of human TACI or amino acids 139-152 of murine TACI as described above.

This invention also does not include compositions of matter consisting only or essentially of: native E3/6.7K protein, native human and murine TACI, known proteins identified at items [2]-[10], [12] and [13] above or known fragments of these proteins. However, this invention includes compositions of matter consisting of the CAML-binding peptides, sequences and motifs of this invention, as defined herein.

This invention includes an isolated CAML-binding peptide comprising a sequence of amino acids defined by the motif:

C-C-x(2)-[FILV]-[ACV]-x(2)-[CS]-x(3)-[KR]-[KR], wherein: x represents any amino acid; a bracketed numeral represents the number of or a range of numbers of any amino acid represented by x; a single letter represents a specific amino acid identified by standard single letter amino acid code; and, a bracketed set of two or more single letters represents alternate amino acids at a single position; providing that the ligand does not comprise more than 100 contiguous amino acids of native TACI.

This invention provides for methods for targeted binding of a moiety or ligand to CAML, including for modulation of immune response and for modulation of apoptosis, which methods make use of the CAML-binding peptides of this invention.

This invention provides a method for binding a ligand to CAML, comprising combining CAML with a ligand, the ligand comprising a peptide defined by the amino acid motif:

C-C-x(2)-[FILV]-[ACV]-x(2)-[CS]-x(3)-[KR]-[KR], wherein: x represents any amino acid; a bracketed numeral represents the number of or a range of numbers of any amino acid represented by x; a single letter other than x represents a specific amino acid identified by standard single letter amino acid code; and, a bracketed set of two or more single letters represents alternate specific amino acids at a single position; providing that the ligand does not comprise more than 100 contiguous amino acids of native TACI.

This invention also provides nucleic acids and nucleic acid vectors encoding the CAML-binding peptides of this invention, for use in treatment (such as gene therapy or to minimize transplant rejection), and for use in recombinant expression of the peptides of this invention or proteins comprising the peptides of this invention. Also included are cells comprising nucleic acids and vectors according to this invention.

This invention provides methods for effecting targeted binding of a ligand or other moiety to CAML, including for inhibiting or inducing apoptosis or treatment of inflammation comprising treating the cell, a mammal comprising the cell, or a tissue comprising the cell, with a medicament comprising a CAML-binding peptide of this invention. The treating may comprise administering a nucleic acid encoding a peptide of this invention whereby the peptide is expressed in the cell. The administering may be by a viral vector comprising the nucleic acid, with the proviso that if the vector is adenovirus or myxoma virus, the nucleic acid is other than a naturally occurring nucleic acid from E3 of adenovirus, or the nucleic acid is under the transcription control of a promotor not found in the selected virus.

The methods of this invention may be employed for treatment of a mammalian patient suffering from a degenerative (e.g. neurodegenerative) disease, an immunodeficiency, or an inflammatory disease by modulation of immune response or apoptosis.

This invention also provides for methods of modulating immune response or programmed cell death in a tissue or cell population in a patient comprising: (a) withdrawing tissue or a cell from the patient, (b) treating the tissue or cells with an apoptosis modulating moiety comprising a CAML-binding peptide of this invention; and (c) returning the treated tissue or cells to the patient.

This invention also provides medicaments, pharmaceutical compositions and medical devices comprising a CAML binding peptide of this invention, a carrier suitable for facilitating delivery of the peptide to a cell and optionally other active components such as a moiety intended to affect CAML activity. Also provided is a nucleic acid comprising a non-naturally occurring adenovirus E3 nucleic acid capable of encoding a CAML-binding peptide of this invention.

This invention also provides recombinant virus comprising a nucleic acid encoding a CAML-binding peptide of this invention with the proviso that if the virus is adenovirus or myxoma virus, the nucleic acid is other than a naturally occurring adenovirus E3 nucleic acid or myxoma virus nucleic acid; or, the nucleic acid is under the transcriptional control of a promoter not from the selected virus.

This invention also provides: the use of a CAML-binding peptide of this invention, a nucleic acid encoding said peptide or a vector comprising said nucleic acid, for treatment, including for inducing or inhibiting apoptosis, for modulating an immune or inflammatory response; and, the use of a CAML-binding peptide of this invention, a nucleic acid encoding said peptide or a vector comprising said nucleic acid for the preparation of a medicament, pharmaceutical composition or implantable medical device or material, for such uses.

This invention also provides an assay for selecting an agent capable of modulating activity of CAML which comprises: combining a CAML-binding peptide with a sample suspected of comprising such an agent or with a putative agent; and, determining whether such CAML activity is modulated. Determining whether CAML activity is modulated may be done by known means for assessing CAML activity.

Combining of CAML and a CAML-binding peptide may occur in vitro (e.g. in a cell extract) or may be done in vivo, as in a cell that is a cell that is rescued from apoptosis or an immune response. The combining in an assay of this invention may include a coupling of a peptide of this invention to an agent or putative CAML modulating agent.

### BRIEF DESCRIPTION OF THE DRAWINGS

FIG. 1 A chart showing alignment of a nucleic acid sequence which is capable of encoding a E3/6.7K protein corresponding to that of Adenovirus serotype Ad2 wild-type(Wt.) (SEQ ID NO:20); and the polymerase chain reaction (PCR) nucleic acid product expected (SEQ ID NO: 21) when a forward primer (FP—SEQ ID NO: 22) and a reverse primer (RP—SEQ ID NO: 23) are used to amplity the wild-type sequence(Wt.). Start codons are underlined. The nucleic acids shown in bold in the forward primer (FP) represent a modification to provide a Kozak consensus sequence. The nucleic acids shown in bold in the reverse primer (RP) is a modified stop codon to enhance translation.

FIG. 2 A chart showing alignment of E3/6.7K amino acid sequences from the Ad2 (SEQ ID NO: 24) and Ad5 (SEQ ID NO: 25) Adenovirus serotypes. The Ad2 E3/6.7K amino acid sequence is 61 amino acids in length and the Ad5 E3i6.7K amino acid sequence is 63 amino acids in length.

### DETAILED DESCRIPTION OF THE INVENTION

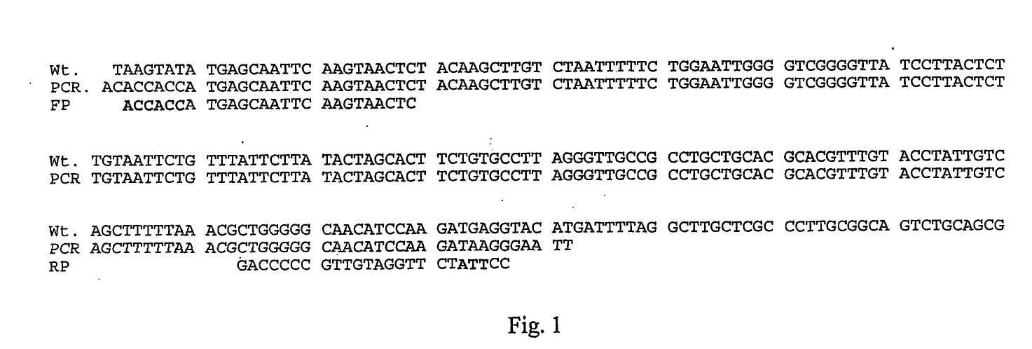

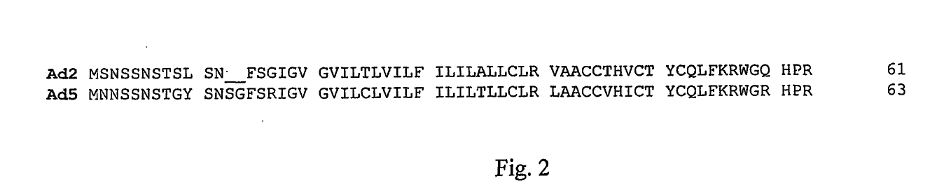

As used herein for description of this invention, the terms E3/6.7K protein, peptide or polypeptide includes a protein or fragment thereof encoded by a nucleic acid as depicted in FIG. 1 or an Ad2 or Ads adenovirus serotype protein or fragment thereof as depicted in FIG. 2. The Ads protein shown in FIG. 2 is about 7.1K.

Modulation of apoptosis, including inhibition of apoptosis or rescue of a cell from apoptosis may be determined by various methods known in the art, including assays which directly measure apoptosis or which measure the activity of TNF-α, such as those described herein.

Recombinant Expression and Gene Therapy Methods. The isolated nucleic acid molecule depicted in FIG. 1, a nucleic acid molecule encoding a CAML-binding peptide as defined herein or a nucleic acid molecule complementary to those described above, may be incorporated into a vector suitable for expression in a host cell or to act as a transformation vector, such as to introduce the nucleic acid into cells of a mammal to be treated. Suitable vectors for such purposes include retroviruses and adenoviruses.

Techniques for the formation of the transfection vector comprising a CAML-binding peptide encoding nucleic acid molecule are well-known in the art, and are generally described in “Working Toward Human Gene Therapy,” Chapter 28 in Recombinant DNA, 2nd Ed, Watson, J. D. ci al., eds., New York: Scientific American Books, pp. 567-581 (1992), and in the references cited therein. Various promoters may be used to enhance expression in host cells or gene expression in specific tissues. For example, in neuronal tissue the neuron-specific enolase promoter (Ad-NSE) and in Lymphocytes the lck promoter could be used for gene therapy methods.

Organ Transplant Methods. CAML-binding peptides have potential uses in tissue and organ transplantation, for example, to render them less susceptible to apoptosis. In particular, they can be used to genetically modify endothelial or other mammalian cells to render them capable of expressing a protein which binds to and is designed to specifically inhibit apoptosis (e.g. as induced by TNF-α) in transfected cells. Peptides of this invention may also be used in the transplantation of genetically modified cells, or tissue or organs comprising such cells, capable of expressing the inhibiting protein; it most particularly is directed to methods of transplanting modified xenogeneic or allogeneic cells, tissue or organs; recombinant genes, proteins and vectors for accomplishing same; and the cells, tissue or organs, as well as non-human transgenie or somatic recombinant animals, so modified.

Appropriate methods of inserting foreign cells or nucleic acids into animal tissue include microinjection, embryonic stem (ES) cell manipulation, electroporation, cell gun, transfection-k, transduction, retroviral infection, etc. Nucleic acids can be inserted into germ cells (e.g. fertilized ova) to produce transgenic non-human animals bearing the gene, which is then passed on to offspring. Nucleic acids can also be inserted into somatic/body cells to provide somatic recombinants, from whom the gene is not passed on to offspring. Transcription of DNA may be made subject to an inducible promoter, so that expression of a recombinant protein can be delayed for a suitable period of time prior to grafting. DNA may be inserted into a particular locus, e.g. the thrombomodulin or P-selectin locus and the construct introduced into embryonic stem (ES) cells, with the resulting progeny expressing the construct in vascular endothelium. Retroviral vectors, and in particular replication-defective retroviral vectors lacking one or more of the gag, pol, and env sequences required for retroviral replication, are well-known to the art and may be used to transform endothelial cells. The ability of adenoviruses to attach to cells at low ambient temperatures is an advantage in the transplant setting which, can facilitate gene transfer during cold preservation.

Alternative means of targeted nucleic acid delivery comprise DNA-protein conjugates, liposomes, etc.

Cells or cell populations can be treated in accordance with the present invention in vivo or in vitro. For example, for purposes of in vivo treatments, p65RHD vectors can be inserted by direct infection of cells, tissues or organs in situ. For example, the vessels of an organ such as a kidney can be temporarily clamped off from the blood circulation, and the blood vessels perfused with a solution comprising a transmissible vector construct containing a CAML-binding peptide for a time sufficient for the gene to be inserted into cells of the organ; and on removal of the clamps, blood flow can then be restored to the organ and its normal functioning resumed.

Cell modification can be carried out ex vivo. Cell populations can be removed from the donor or patient, genetically modified by insertion of a vector, and then implanted into the patient or a syngeneic or allogeneic recipient. For example, an organ can be removed from a donor, subjected ex vivo to the perfusion step described above, and the organ can be re-grafted into the donor or implanted into a different recipient of the same or different species.

Preferably, DNA encoding a peptide of this invention will be placed under the control of a constitutive or inducible promoter. An advantage of employing an inducible promoter for transplantation purposes is that the desired high level transcription/expression of the active gene/protein can be delayed for a suitable period of time before grafting. For example, transcription can be obtained on demand in response to a predetermined stimulus, such as, e.g. the presence of tetracycline in the cellular environment. An example of a tetracycline-inducible promoter which is suitable for use in the invention is disclosed by Furte et al., PEAS US 91 (1994) 9302-9306. Alternatively, a promoter system where transcription is initiated by the withdrawal of tetracycline is described by Gossen and Bujard, PEAS URSA 90 (1992) 5547-51.

Peptide preparation, expression and administration. A peptide according to the invention or a derivative thereof such as a chimeric peptide (including proteins) comprising a CAML-binding peptide may be administered as a pharmaceutical composition which may be formulated according to various methods. For example, such a formulation may be a solution or suspension However, as is well known, peptides can also be formulated for therapeutic administration as tablets, pills, capsules, sustained release formulations or powders. The preparation of therapeutic compositions which comprise polypeptides as active ingredients is well understood in the art. Typically, such compositions are prepared in injectable form, e.g. as liquid solutions or suspensions.

Peptides to be used according to this invention may be synthesized using standard techniques such as those described in Bodansky, M. Principles of Peptide Synthesis (1993) Springer Verlag, Berlin. Automated peptide synthesizers are commercially available (e.g. Advanced ChemTech Model 396; Milligen/Biosearch 9600). Peptides may be purified by high pressure liquid chromatography and analyzed by mass spectrometry. One or more modifying groups may be attached to such a peptide by standard methods, for example by modification of amino, carboxyl, hydroxyl or other suitable reactive groups on an amino acid side chain or at either terminus of a peptide (e.g. Greene, T. W. and Wuts, P. G. M. Protective Groups in Organic Synthesis (1991) John Wyley & Sons Inc., New York). Peptides may also be prepared according to standard recombinant techniques using a nucleic acid molecule encoding the peptide. A nucleotide sequence encoding a desired peptide may be determined pursuant to the genetic code and an oligonucleotide having this sequence may be synthesized by standard DNA synthesis methods (e.g. using automated DNA synthesizer) or by deriving such DNA from a natural gene or cDNA using standard molecular biology techniques such as site-directed mutagenesis, polymerase chain reaction, and/or restriction enzyme digestion. For example, production of recombinant adenovirus and TACI proteins is known in the art, including from literature described herein.

To facilitate expression of a peptide in a host cell by recombinant techniques, nucleic acids according to this invention may be incorporated into a recombinant vector. Accordingly, this invention also provides such vectors comprising the nucleic acid molecules of this invention. As used herein, the term “vector” refers to a nucleic acid molecule capable of transporting another nucleic acid to which it has been linked. Vectors may include circular double stranded plasmids and viral vectors. Certain vectors are capable of autonomous replication in a host cell such as vectors of bacterial origin and episomal mammalian vectors. Other vectors such as non-episomal mammalian vectors may be integrated into the genome of a host cell upon introduction into the host cell and thereby may be replicated along with the host cell genome. Certain vectors may be capable of directing the expression of genes to which they have been operatively linked and are referred to as expression vectors.

A nucleotide sequence encoding a peptide of or to be used in this invention may be operatively linked to one or more regulatory sequences selected on the basis of the host cells to be used for expression. This means that the sequences encoding the peptide are linked to a regulatory sequence in a manner that allows for expression of the peptide. Such regulatory sequences may include promoters, enhancers, polyadenylation signals and other expression control elements such as are described in Goddel; Gene Expression Technology: Methods in Enzymology 185 (1990) Academic Press, San Diego, Calif. Regulatory sequences may direct constituative expression in many types of host cells or may direct expression only in certain tissues or cells. Regulatory elements may direct expression in a regulatable manner such as only in the presence of an inducing agent. Suitable expression vectors for adenovirus proteins and for TACI are known in the art, including references referred to herein.

Peptides, polypeptides and proteins to be used according to this invention may comprise sequences of amino acids not derived from the natural source for a dedicated CAML-binding peptide employed (e.g. fusion or chimeric protein). For example, such proteins may comprise a peptide of this invention fused to a peptide that facilitates transfer across a cell membrane or fused to a peptide that affects or facilitates modulation of CAML activity. Also included in this invention are derivatives of peptides of this invention, including derivatives intended to enhance the immunogenicity, biological activity, or pharmacokinetic properties of the peptide or protein comprising the peptide. Further, peptides of this invention may be modified by labeling or by coupling to another agent intended to facilitate detection or recovery of the peptide or its binding partners, including CAML. Examples of such labeling include coupling to an enzyme or a detectable label such as a metallic, radioactive, or fluorescent element. Examples of modification to affect pharmacokinetic properties include modification of N or C termini (e.g. to include an amide group or a D-amino acid) to reduce the ability of a peptide to act as a substrate for a carboxypeptidase or a aminopeptidase, or myristoylation to improve accessibility to a cell interior.

Examples of suitable parenteral administration include intravenous, subcutaneous and intramuscular routes. Intravenous administration can be used to obtain acute regulation of peak plasma concentrations of the drug as might be needed for example to treat acute episodes of airway hyper-responsiveness. Improved half-life and targeting of the drug to the airway epithelia may be aided by entrapment of the drug in liposomes. It may be possible to improve the selectivity of liposomal targeting to the airways by incorporation of ligands into the outside of the liposomes that bind to airway-specific macromolecules. Alternatively intramuscular or subcutaneous depot injection with or without encapsulation of the drug into degradable microspheres e.g. comprising poly (DL-lactide-co-glycolide) may be used to obtain prolonged sustained drug release as may be necessary to suppress the development of airway hyper-responsiveness. For improved convenience of the dosage form it may be possible to use an i.p. implanted reservoir and septum such as the Percuseal system available from Pharmacia Improved convenience and patient compliance may also be achieved by the use of either injector pens (e.g. the Novo Pin or Q-pen) or needle-free jet injectors (e.g. from Bioject, Mediject or Becton Dickinson). Prolonged zero-order or other precisely controlled release such as pulsatile release can also be achieved as needed using implantable pumps. Examples include the subcutaneously implanted osmotic pumps available from ALZA, such as the ALZET osmotic pump.

Nasal delivery may be achieved by incorporation of the protein drug into bioadhesive particulate carriers (<200 μm) such as those comprising cellulose, polyacrylate or polycarbophil, in conjunction with suitable absorption enhancers such as phospholipids or acylcamitines. Available systems include those developed by DanBiosys and Scios Nova.

Oral delivery may be achieved by incorporation of a drug into enteric coated capsules designed to release the drug into the colon where digestive protease activity is low. Examples include the OROS-CT/Osmet.TM. and PULSINCAP.TM. systems from ALZA and Scherer Drug Delivery Systems respectively. Other systems use azo-crosslinked polymers that are degraded by colon specific bacterial azoreductases, or pH sensitive polyacrylatc polymers that are activated by the rise in pH at the colon. The above systems may be used in conjunction with a wide range of available absorption enhancers.

Targeted delivery of high doses of a drug to the site of airway hyper-responsiveness can be directly achieved by pulmonary delivery (see: McElvaney, et al., J. Clin. Invest., 90, 1296-1301 (1992); and Vogelmeier et al., J. Appl. Physiol., 69, 1843-1848 (1990). The lower airway epithelia are highly permeable to wide range of proteins of molecular sizes up to 20 kDa (e.g. granulocyte colony stimulating factor). It is possible to spray dry proteins in suitable carriers such as mannitol, sucrose or lactose. Micron-sized particles may be delivered to the distal alveolar surface using dry powder inhalers similar in principle to those designed by Inhale, Dura, Fisons (Spinhaler), Glaxo (Rotahaler) or Astra (Turbohaler) propellant-based metered dose inhalers. Solution formulations with or without liposomes may be delivered using ultrasonic nebulizers.

Examples of inflammation caused by implants or surgical procedures, the treatment of which may make use of this invention include: restenosis, senile calcific aortic stenosis, balloon angioplasty induced inflammation, intimal hyperplasia (a cause of vascular restenosis), atherosclerosis, benign prostate hyperplasia, hysteroscopically delivered fallopian tube fertilization and sterilization aided therapies, inflammation caused by catheters, inflammation caused by the use of mesh or other implants for hernia repair, inflammation caused by the use of mesh in the surgical repair of rectocoele and rectal prolapses, urological stress, incontinence, inflammation caused by surgical uterine suspensions, inflammation caused by tapes, staples and sutures, and inflammation resulting from vascular grafts (including peripheral, coronary artery and bypass grafts). In such cases, treatment may include provision of peptides or nucleic acids of this invention on an implantable medical device including beads, tape, mesh, gauze, membranes, appliances such as stents, and the like. Methods for associating (e.g. coating) of peptides or nucleic acids with such devices or materials are known, including those described in: WO 98/16268, WO 00/23123, U.S. Pat. No. 6,140,128, U.S. Pat. No. 4,610,692, U.S. Pat. No. 6,117,456, U.S. Pat. No. 4,946,686, Tarr, E. R. (1997) Biomed Sci Instrum, 33:143-8, and Abrams, L. (1994) Biomed Sci Istrum, 30:169-74.

Percutaneous transluminal coronary angioplasty (PTCA) is widely used as the primary treatment modality in many patients with coronary artery disease, to reduce obstruction and improving coronary flow. However, restenosis often results with significant morbidity and frequently necessitates fierier interventions such as repeat angioplasty or coronary bypass surgery.

The present invention includes therapeutic methods comprising the administration of a therapeutic agent comprising or in addition to a CAML-binding protein of this invention to a procedurally traumatized mammalian vessel (e.g., by an angioplasty procedure). Preferably, the therapeutic agent is an E3/6.7K or TACI derived peptide or polynucleotide of this invention. Preferred therapeutics in the practice of the present invention, include, for example The SED domain of E3/6.7K or analogs or derivatives thereof. The administration of a therapeutic agent of the invention is effective to biologically stent the vessel, inhibit or reduce vascular remodeling of the vessel, inhibit or reduce vascular smooth muscle cell proliferation, or any combination thereof. The administration of the therapeutic agent preferably is carried out during the procedure, which traumatizes the vessel, e.g., during the angioplasty or other vascular surgical procedure. The invention also provides therapeutic compositions and dosage forms adapted for use in the present method, as well as kits containing them.

Thus, this invention includes methods for biologically stenting a traumatized mammalian blood vessel. The method comprises administering to the blood vessel an effective amount of a therapeutic agent to biologically stent the vessel. As used herein, “biological stenting” means the fixation of the vascular lumen in a dilated state near its maximal systolic diameter, e.g., the diameter achieved following balloon dilation and maintained by systolic pressure. The method may comprise the administration of an effective amount of an E3/6.7K peptide or polynucleotide into the blood vessel. Preferably, the peptide or polynucleotide is dispersed in a pharmaceutically acceptable liquid carrier, viral vector or liposome mediated gene delivery preferably administered locally via a catheter. The peptide or polynucleotide may be dispersed in a pharmaceutically acceptable liquid carrier at about 0.001 to about 25 mu.g per ml of aqueous vehicle. Preferably, a portion of the amount administered penetrates to at least about 6 to 9 cell layers of the inner tunica media of the vessel and so is effective to biologically stent the vessel.

The invention also includes therapeutic methods comprising inhibiting diminution of vessel lumen diameter by administering to a traumatized vessel of a mammal an effective amount of a CAML-binding peptide. For example, an E3/6.7K or TACI derived peptide or polynucleotide is administered via an implantable device, wherein the implantable device is not a catheter, which has a first and a second expansile member, i.e., balloons, which are disposed on opposite sides of the vessel area to be treated in order to isolate the portion of the vessel to be treated prior to peptide or polynucleotide administration. Preferably, the isolated portion of the vessel is not washed to remove blood prior to peptide or polynucleotide administration (“bloodless angioplasty”). “Isolated,” as used above, does not mean occlusive contact of the actual treatment area by the catheter balloon, which is preferred in the practice of the present invention.

The invention further includes methods for inhibiting or reducing diminution in vessel lumen volume in a traumatized mammalian blood vessel. The method may comprise administering to the blood vessel of a mammal an effective amount of peptide or polynucleotide of this invention, wherein the peptide or polynucleotide is in sustained release dosage form. Preferably, the peptide or polynucleotide is administered in situ, by means of an implantable device, wherein the peptide or polynucleotide is releasably embedded in, coated on, or embedded in and coated on, the implantable device. A crystalline peptide may be releasably embedded in, or dispersed in, a adventitial wrap, e.g., a silicone membrane.

The invention further includes therapeutic methods comprising administering to a traumatized mammalian blood vessel a sustained release dosage form comprising microparticles or nanoparticles comprising a peptide or polynucleotide of this invention or analogs thereof For example, a sustained release dosage form comprising a SED peptide is preferably administered via an implantable device, which is not a catheter used to perform bloodless angioplasty. The amount administered will be effective to inhibit or reduce diminution in vessel lumen area of the mammalian blood vessel. The sustained release dosage form preferably comprises microparticles of 4 to about 50 microns in diameter. The sustained release dosage form can also preferably comprise about 2 to about 50, and more preferably greater than 3 and less than 10, microns in diameter. For nanoparticles, preferred sizes include about 10 to about 5000, more preferably about 20 to about 500, and more preferably about 50 to about 200, nanometers.

Also included are methods comprising administering to a mammalian blood vessel a dosage form of peptide of this invention or an analog thereof in a non-liquid vehicle or matrix effective inhibit or reduce diminution in vessel lumen area of the mammalian blood vessel. Preferably the dosage form is a substantially solid dosage form. As used herein, “solid form” does not include microparticles, nanoparticles, and the like. The non-liquid vehicle or matrix preferably includes, but is not limited to, a gel, paste, gauze or a membrane, which comprises the peptide.

Also included is a kit comprising, preferably separately packaged, at least one implantable device adapted for the in situ delivery of at least one peptide or polynucleotide of this invention to a site in the lumen of a traumatized mammalian vessel and at least one unit dosage form of a therapeutic agent comprising the peptide or polynucleotide in a liquid vehicle adapted for delivery by said device. The administration of at least a portion of the unit dosage form to the traumatized vessel is intended to be effective to biologically stent the vessel, inhibit or reduce the vascular remodeling of the vessel, inhibit or reduce vascular smooth muscle cell proliferation, or any combination thereof.

Further included is a kit comprising, preferably separately packaged, an implantable device adapted for the delivery of at least one therapeutic agent to a site in the lumen of a traumatized mammalian vessel and a unit dosage form comprising at least one peptide or polynucleotide of this invention, wherein the administration of at least a portion of the unit dosage form is effective to cause CAML-binding and actions of the therapeutic agent to inhibit or reduce diminution in vessel lumen diameter of the vessel. The device could be a catheter, having a first and a second expansile member, which are disposed on opposite sides of the region to be treated so as to isolate a portion of the vessel to be treated. Alternatively, the isolated portion of the vessel is not washed to remove blood prior to administration.

The invention also includes pharmaceutical compositions suitable for administration by means of an implantable device. The composition may comprise an amount of a SED peptide or analog thereof effective to inhibit or reduce stenosis or restenosis of a mammalian vessel traumatized by a surgical procedure and a pharmaceutically acceptable non-liquid release matrix for said SED peptide. Preferably, the release matrix comprises a gel, paste, gauze or membrane.

The invention also includes therapeutic devices. One such embodiment comprises a therapeutic shunt comprising an amount of a peptide or polynucleotide of this invention effective to facilitate inhibition of stenosis or reduce restenosis following placement of the therapeutic shunt as a result of CAML interaction. Another embodiment of the invention comprises therapeutic artificial graft comprising an amount of a SED peptide or analog thereof to inhibit stenosis or reduce restenosis following placement of the graft. Yet another embodiment of the invention comprises a therapeutic adventitial wrap comprising an amount of a SED peptide or polynucleotide effective to inhibit stenosis or reduce restenosis following placement of the wrap.

The amount of a pharmaceutical composition according to this invention to be employed will depend on the recipient and the condition being treated. The requisite amount may be determined without undue experimentation by protocols known to those skilled in the art. Alternatively, the requisite amount may be calculated, based on a determination of the amount of tryptase which, must be inhibited in order to treat the condition.

### EXAMPLES

Virus strains and tissue culture. Wild Type Ads (Ad5wt) was obtained from the American Type Culture Collection (Rockville, Md., USA) and dl739, E3/6.7K-deleted viral mutant (dl739) was according to Brady et al. (1992), J. Virol. 66:5914-5923. These two adenovirus group C viruses share a great degree of similarity, but differ in the expression of E3/6.7K protein, which is deleted in dl739. Both viral serotypes were propagated in monolayer culture of A549 cells grown in Minimal Essential Media (Gibco BRL Life Technologies Inc., Gaithersburg, Md., USA) supplemented with 10% Fetal Calf Serum (FCS). Two to five days after inoculation with Ad5, cells were freeze/thawed twice, sonicated for 30s three times and centrifuged at 500×g for 5 min. The supernatant was collected and its viral titer determined by plaque assays on A549 monolayers grown on six well plates. Titers ranged from 10⁸ to 10⁹ plaque-forming units (pfu)/ml. Control inoculum was prepared from uninfected A549 cells treated in an identical manner to the infected cells.

Inoculation of airway ducts and viral plaque assays. Two groups of 24 mice were anaesthetized with Halothane. One group of mice were infected intranasally with 10⁷ pfu of Adswt in 60 μl of culture media while the other group of mice was infected intranasally with 10⁷ pfu of dl739 in 60 μl of culture media. Six animals were infected with sterile culture media alone. Six animals from each of the two groups were sacrificed with an overdose of Halothane 2 hours, 1, 3 and 7 days post infection (p.i.). Two sham infected animals were sacrificed on days 1, 3 and 7 days pi.. The left lung was removed and frozen in liquid nitrogen for use in viral plaque assays. The right lung was inflated with 4% paraformaldehyde in PBS pH7.4 (0.149 M NaCl, 0.012 M Na2HPO4, 0.004M KH2PO4) and embedded in paraffin.

Viral titer. Viral plaque assays were used to quantitate the amount of replicating virus in mouse lungs. Approximately 200 mg of lung was homogenized on ice in 1 ml sterile MEM with a polytron. The homogenate was spun for 2 min at 10,000 g while the supernatant was removed and stored at −70° C. The lung homogenate supernatant titer was determined by plaque assay on A549 cell monolayer cultures grown in MEMI10% FBS on six well plates using decimal dilutions from 10-1 to 10-6 in MEM from the supernatant of animal lung homogenate from all time groups. Each well was inoculated with 500 μl of diluted supernatant and virus was allowed to adsorb onto the monolayer of A549 cells for 1 h. at 37° C. An agarose overlay (0.9% agarose, MEM, 2% FCS, and 0.001 neutral red at 37° C.) was applied after adsorption. Plaques were counted after 10-14 days and normalized to lung mass and expressed as (log pfu/g lung tissue).

Histologic scoring. Four μm sections of paraffin embedded lung tissue were mounted on glass slides and stained with hematoxylin and eosin. An independent observer scored the airway mucosal, airway adventitia and the vascular adventitia for inflammation. The histopathologic grades were 0—no inflammation, 1—mild inflammation. 2—moderate inflammation, 3—severe inflammation for each feature. The scores for each feature were summed to give a total inflammatory score with maximum being 9 for each animal. A mean inflammatory score was calculated for each animal by dividing the total score by 3. The mean and standard deviation was calculated for each experimental group.

Statistical analysis. Comparisons between the two virus were made for viral titer, inflammatory score and time using a 2-way ANOVA. The level of significance was p<0.05.

Plasmid constructs. CDNA for E3/6.7K was obtained by amplifying by PCR the region coding for the E3/6.7K ORF from a vector carrying the Ad2 E3 region using SEQ ID NO:22 and 23 as primers (FIG. 1). The PCR product was cloned in the Xhol site of the BPV based cDNA expression vector pBCMGSneo (Karasuyama and Melchers, (1988), Eur. J. Immunol. 18:97-104) and sequenced to ensure accuracy.

To isolate the cDNA for E3/6.7K, the primers and template DNA purified from HEK-293 cells infected with Ad2 and AdS, 24 hr. post infection were used. The reaction cocktail contained template DNA, 0.5 μM of each primer forward and reverse, 250 μM of each nucleotide, 5 U of Pfu polymerase (Canadian Life Technologies, 2270 Industrial St., Burlington, Ontario) in 1×Pfu Buffer. The reaction conditions are: melting of double stranded DNA at 95° C. for 30 sec., followed by annealing at 57° C. for 30 sec., followed by a 30 sec. ramp to 72 and continued elongation for an additional 30sec. 30 cycles of the above PCR reaction produced sufficient DNA in most cases. The newly generated cDNA for E3/6.7K contained modifications (highlighted in bold in FIG. 1) which are not found in naturally occurring E3 nucleic acid. Both modifications enhance translation initiation at the start site of E3/6.7K and provide for increased production of the protein in a transformed cell. The forward primer provides the start site of E3/6.7K with an optimal upstream Kozak consensus sequence. The reverse primer was modified to replace the naturally occurring TGA-Stop codon with an Ochre-Stop codon (TAA). The latter modification eliminates the start site of E3/19K, which overlaps with the sequence of E3/6.7K in the naturally occurring E3 nucleic acid and decreases translation efficiency of E3/6.7K from the natural sequence.

pBD-6.7, a bait vector containing E3/6.7, was constructed by subdloning full length E3/6.7K cDNA in frame into the C terminus of the GAL4 DNA binding domain of vector pGBKT7 (CLONTECH). pAD-CAML was constructed by subcloning full length CAML in frame into the C terminus of the GAL4 activation domain of the prey vector pGADT7 (CLONTECH). pAD-CAML(201) was constructed by subdloning amino acids 1 to 201 of CAML in frame into the C terminus of the GAl4 activation domain of the prey vector pGADT7. The fidelity of the constructs was confirmed by sequencing.

Yeast Transformation and Selection. p]3D-6.7 and pAD-CAML or pAD-CAML(201) were transformed together into Saccharomyces cerevisiae AH109 using the Lithium Acetate Yeast Transformation procedure as described by Gietz, D., et al. (1992) Nucleic Acids Res., 20:1425. Control cotransformation of p3D-53 /pAD-T and pBD-Lam/pAD-T were also done. pBD-53/pAD-T encode fusions between the GAL4 DNA-BD and AD and murine p53 and SV40 large T-antigen, respectively. p53 and large T-antigen interact in a yeast two-hybrid assay. pBD-Lam encodes a fusion of the DNA-BD with human lamin C and provides a negative control for an interaction with pAD-T. For yeast selection, pBD containing the ThPI gene was selected on synthetic drop out media lacking tryptophan (SD-T). pAD contains the LEU2 gene and was selected for on SD-L (leucine). When selecting for protein interaction AH109 was grown on leucine/-tryptophan/-histidine deficient/X-a-Gal supplemented media (SD-LTEDI or adenine/-histidine/-leucine/-tryptophan deficient/X-a-Gal supplemented media (SD-AHLTX). The transformed yeast can only grow on SD-LTHX or SD-AHLTX if a protein interaction occurs. Yeast transformed with pBD-6.7 and pAD-CAML or pBD-6.7 and pAD-CAML(201) were able to grow on both SD-LTHX or SD-AHLTX indicating that the reporter enzymes necessary to grow on deficient media are synthesized which indicates that the proteins E3/6.7K and CAML interact.

Generation of stable U937 cell lines expressing E3/6.7K U937 human histiocytic lymphoma cells obtained from ATCC (CRL 1593) were maintained in RPMI 1640, 10% FCS, 2 mM L-glutamine, 10 mM HEPES, 100 U/ml penicillin and 100 μg/ml streptomycin in an atmosphere of 5% CO2 and 100% humidity. Cells were transfected with the appropriate construct by using the DMRIE-C cationic lipid reagent available from Life Technologies using the manufacturer's protocol. Transfected cells were maintained in medium containing geneticin G-418 sulphate at a final concentration of 800 μg/ml. Media and supplements were purchased from Life Technologies. Subclones of the transfected cell lines were generated by serial dilution and examined for expression of E3/6.7K by Northern Blotting. The expression of E3/6.7K mRNA was very similar in all the clones examined. All the G-418 resistant cells that survived the selection procedure were pooled and used for the in vitro assays, in order to avoid clonal variations known to arise in U937 cells.

Labelling, immunoprecipitation and Western Blotting of proteins from transfected cells. U937 cells transfected with vector or with vector carring E3/6.7K were grown in suspension until they were growing exponentially. 10⁸ cells were harvested, washed and intracellular pools of cysteine and methionine were depleted by incubation in prewarmed methionine/cysteine-free essential media without FCS for one hour at 37° C. at a concentration of 5×10⁶ cells/ml. A total of 2×10⁷ cells were labelled for one hour in prewarmed methionine/cysteine-free media containing 0.5 mCi/ml [35S]-Cysteine and 0.2 mCi/ml (Amersham) [35S]-Methionine (Amersham) at a concentration of 5×10⁶ cells/ml. Cells were washed and then lysed on ice in freshly made lysis buffer containing 1% TritonX-100, 1% BSA (bovine serum albumin), 1 mM iodoacetamide, 1 mM PMSF, 2.5 TIU/ml aprotinin, 0.01M Tris pH8.0, 0.14M NaCl. Samples were counted by TCA precipitation and approx. 10⁷ cpm of each sample was precleared O/N using protein A-Sepharose CL-4B, the supernatant was immunoprecipitated using a polyclonal rabbit antiserum raised against the C-terminal portion of E3/6.7K and protein A-Sepharose. The pellet was denatured in SDS/sample buffer and loaded on a Tricine-SDS PAGE gel, 16.5% T, 3% C separating gel with a 10% T, 3% C spacer gel (Schagger and von Jagow, 1987). Alternatively, cell lysate equivalent to 105 cells was denatured in SDS-PAGE loading buffer and loaded on 10% glycine SDS-PAGE gel system, separated and blotted onto a Immobilon-P PVDF membrane (Millipore) and probed with cPLA2 rabbit polyclonal antiserum (Cayman Chemical). The signal was detected via horse radish peroxidase-conjugated, goat antirabbit antiserum and by chemiluminescence using the ECL kit (Biorad).

Arachidonic acid release assays. Cells were grown at low density in 10% Hyclone FCS, RPMI 1640, 2 mM L-glutamine, 10 mM HEPES for several days then harvested and washed twice in PBS, 1% BSA. Approximately 5×10⁶ cells (5×10⁵ cells/ml) were labelled for 20 hrs in same media as above supplemented with 0.4 μCi/ml [3H] arachidonic acid [5,6,8,9,11,12,14,15-3H(N)] (0.2 mCi/ml stock; New England Nuclear). Cells were washed twice in RPMI 1640, 0.2%BSA and incubated for one hour in the wash media in order to minimize the spontaneous release of [3H] arachidonic acid. The number of cells was normalized in all cell lines and 400 μl of cell suspension was aliquoted in each well of a 24 well plate containing 100 μl of treatment media (2×10⁵ cells/well corresponding to 1.4×10³ counts/well). The assay was set up in triplicate and the cells were stimulated either with media alone or with 20 ng/ml human rTNF-α (2000 U/ml) (Boehringer, Mannheim), or with 10 μg/ml cycloheximide or with a combination of 20 ng/ml TNF-A and 10 μg/ml cycloheximide. After 20 hours of treatment the cells were centrifuged and 100 μl of supernatant out of 500 μl total was mixed with 3 ml scintillation fluid and counted. For each cell line three samples were lysed in lysis buffer and the lysate was used to determine the total counts of incorporated [3H] Arachidonic Acid. The counts per minute of released [3H] arachidonic acid were expressed as a percentage of the average of total incorporated [3H] arachidonic acid.

Annexin V-FACS apoptosis assay. Annexin V-FITC (PharMingen) was used to determine the binding of Annexin V to externalized phosphatidyl serine. The protocol followed was based on the manufacturers Annexin V-FITC staining protocol. Cells were grown at low density in 10% Hyclone FCS, RPMI 1640, 2 mM L-glutarine, 10 mM HEPES for several days then 5×10⁶ cells were harvested and washed twice in PBS. Cells resuspended in above media were treated for 7 hours with media alone or with 100 ng/ml (10,000 U/ml) human rTNF-α or with 200 μg/ml cycloheximide or with a combination of 100 ng/ml TNF-α and 200 μg/nl cycloheximide. The cells were resuspended at 1×10⁶ cells/ml in 1× Binding Buffer (10 mMHepes/NaOH, pH7.4, 140 mM NaCl, 2.5 mM CaCl2). 1×10⁵ cells (100 μl of above suspension) were combined with 5 μl of Annexin V-FITC. One sample of cells was not stained and used to set up the baseline fluorescence. The cells were examined with a fluorescence-activated cell sorter SACS) on a Beckton Dickson FACS Analyzer.

Production of Ad vectors for gene therapy. The SV5 backbone previously described (Chen (1997) PNAS) has been successfully used to transduce in vivo the dystrophin gene. The backbone lacks the E1 and E2 region. Without these two regions the SV5 Ad vector is replication defective and therefore safer to use as well as it elicits a reduced inflammatory response. The cDNA encoding E3/6.7K under the control of the actin promoter and the CMV enhancer was added to SV5 and used to rescue a new vector called SV5-6.7, which will incorporate E3/6.7K as an immunomodulatory protein.

Creation of Producer Cells resistant to apoptosis. Creation of hybridoma, Chinese hamster ovary (CHO) or insect cells that are resistant to apoptosis follow the same procedure as the transfection of U937 cells outlined above except at the end of the selection in G418, the cells are sorted or clonally expanded in order to screen for the expression of the protein of interest.

E3/6.7K Results in more Persistent Viral Titers and a reduction of the inflammatory response. The presence of E3/6.7K results in more persistent viral titers during the course of infection by comparing mice infected with the E3/6.7K deletion virus (dl739) to mice infected with the wild type virus (Ad5wt). The titers of dl739 were significantly higher than Ad5 wild type one day after inoculation (p<0.001). Over time, the titers of dl739 decreased as the virus was cleared (p<0.001). In contrast Ad5wt titers did not change significantly over the 7 day experimental period. The rapid reduction of dl739 over the seven day period is attributed to a strong host response due to the increased inflammation in the absence of E3/6.7K. Inflammation of the perivascular region of the blood vessels and the adventitia of the airways was greater in animals infected with dl739 than in animals infected with Ad5wt over the seven days experimental period (p=0.025).

There was also a significant increase in inflammation from day three to day seven for both types of viruses (p=0.029).

TNF-α Mediated Arachidonic Acid Release Is Reduced in the Presence of E3/6.7K E3/6.7K can affect the cellular response to inflammatory cytokines. A U937 cell line was transfected with the cDNA for E3/6.7K and expression of E3/6.7K was confirmed using immunoprecipitation with a polyclonal rabbit antiserum raised against an E3/6.7K C-terminal derived peptide and SDS-PAGE electrophoresis. The U937 cells transfected with E3/6.7K cDNA (U937-E3/6.7K) decreased [3H] arachidonic acid release by 50% when compared with U937 cells transfected with vector alone (U937neor) when stimulated with TNF-α. When the stimulus was increased by the addition of TNF-α and cycloheximide (CHX), a protein synthesis inhibitor synergistic with TNF-α, U937-E3/6.7K were still able to reduce the release of [3H] arachidonic acid by 60% when compared to U937neor. The presence of E3/6.7K reduces the levels of inducible release of [3H] arachidonic acid during TNF-α stimulation.

Apoptosis Induced by TNF-α is Reduced in the Presence of E3/6.7K. TNF-α induced apoptosis was assayed by measuring by measuring the externalization of phosphatidyl serine using FITC labelled Annexin V according to Martin et al., (1995), J. Exp. Med. 182:1545-1556. Cells expressing E3/6.7K show a 55% reduction in percentage of apoptotic cells compared with U937neor following stimulation with TNF-α. The U937-E3/6.7K cells show a 65% reduction in apoptosis compared to U937neor following an augmented stimulation with a combination of TNF-α and CHX. The presence of E3/6.7K decreased the apoptotic response in U937 cells upon stimulation with TNF-α or a combination of TNF-α and CHX.

In the Presence of E3/6.7K, cPLA2 Is Cleaved to a 78 kDa Form Following TNF-α Induction. The expression of cPLA2 in U937 cells following induction with TNF-α was assayed. The cPLA2 antiserum recognized two forms of the enzyme: one larger form of approximately 110 kDa; and a second form of 78 kda. There was a difference between U937neor cells and U937-E3/6.7K with regards to the ratio of the 110 kDa versus the 78 kDa forms of cPLA2. While TNF-α does not seem to alter this ratio in U937neor (cells where the predominant form migrates as a 110 kDa protein) in U937-E3/6.7 K cells following induction with TNF-α the most predominant form of cPLA2 is 78 kDa. The antisera was raised against a peptide corresponding to residues 443-462 of the cPLA2 sequence, therefore the only fragment detected by immunoblotting following cleavage is the 78 kDa fragment corresponding to the 1-522 amino acid sequence of cPLA2 as isolated from U937 cells according to Sharp et al. (1991), J. Biol. Chem., 266:14850.

CAML-Binding with SED. The yeast two hybrid system takes advantage of the GAL4 transcriptional activator which consists of two separable and functionally essential domains: a DNA binding domain and a domain that activates transcription. A bait gene (fragments of the E3/6.7K gene) was expressed as a fusion to the GAL4 DNA-binding domain (ED), while a prey gene (CAML) was expressed as a fusion to the GAL4 activation domain (AD). When bait and prey fusion proteins interact, the BD and AD are brought into proximity, thus activating transcription of reporter genes. Using this methodology, it is demonstrated that E3/6.7 K interacts with full length human and mouse CAML and more particularly, with the same domain of CAML that TACI has been previously shown to interact (the 1-201 residue domain of CAML).

Although various aspects of the present invention have been described in detail, it will be apparent that changes and modification of those aspects described herein will fall within the scope of the appended claims. It will be readily apparent to one of ordinary skill in the relevant arts that other suitable modifications and adaptations to the methods and applications described herein are obvious and may be made without departing from the scope of the invention or any embodiment thereof. All publications and patent documents referred to herein are incorporated by reference.

What is claimed is:

1. A method for binding a ligand to CAML, comprising combining CAML with a ligand, the ligand comprising a peptide defined by the amino acid motif:
C-C-x(2)-[FILV]-[ACV]-x(2)-[CS]-x(3)- -R][KR], wherein: x represents any amino acid; a bracketed numeral represents the number of or a range of numbers of any amino acid represented by x; a single letter other than x represents a specific amino acid identified by standard single letter amino acid code; and, a bracketed set of two or more single letters represents alternate specific amino acids at a single position;
providing that the ligand does not comprise more than 100 contiguous amino acids of native TACI.

2. The method of claim 1, wherein the ligand does not comprise more than 75 contiguous amino acids of native TACI.

3. The method of claim 1, wherein the ligand does not comprise more than 50 contiguous amino acids of native TACI.

4. The method of claim 1, wherein the ligand does not comprise more than 40 contiguous amino acids of native TACI.

5. The method of claim 1, wherein the ligand does not comprise more than 30 contiguous amino acids of native TACI.

6. The method of any one of claims 1-5, further providing that the ligand does not comprise native E3/6.7K.

7. The method of claim 6, wherein the ligand comprises a part of native E3/6.7K.

8. The method of any one of claims 1-7, wherein the motif is:
C-C-x(2)-[ILV]-[ACV]-x(2)-[CS]-x(3)-[KR]-[KR]; wherein x, bracketed numerals, letters and bracketed letters are as defined.

9. The method of any one of claims 1-7, wherein the motif is:
C-x(3,5)-C-C-x(2)-[FILV]-[ACV]-x(2)-[CS]-x(3)-[KR]-[R; wherein x, bracketed numerals, letters and bracketed letters are as defined.

10. The method of any one of claims 1-7, wherein the motif is:
L-x(2,3)-C-x(4,5)-C-C-x(2)-[ILV]-[ACV]-x(2)-[CS]-x(3)-[KR]-[KR]; wherein x, bracketed numerals, letters and bracketed letters are as defined.

11. The method of any one of claims 1-7, wherein the motif is:
L-x(1,2)-Cx(5)-C-C-x(2)-[ILV][ACV]-x(2)-[CS]-x(3)-[KR]-[KR]; wherein x, bracketed numerals, letters and bracketed letters are as defined.

12. The method of any one of claims 1-11, wherein the combining is in a cell.

13. The method of claim 12, wherein the peptide is provided in the cell by expressing a nucleic acid encoding the peptide in the cell.

14. The method of any one of claims 1-13, wherein the peptide is selected from the group consisting of SEQ ID NO:1-5.

15. The method of any one of claims 1-13, wherein the peptide is selected from the group consisting of SEQ ID NO:6-19.

16. The method of any one of claims 1-15, wherein the ligand fuler comprises a detectable label.

17. The method of any one of claims 1-15, wherein the ligand modulates CAML activity.

18. The method of any one of claims 1-15, wherein the ligand is a peptide which modulates CAML activity.

19. The use of a peptide for CAML-binding, the peptide defined by the amino acid motif:
C-C-x(2)-[FILV]-[ACV]-x(2)-[CS]-x(3)-R]-[KR], wherein: x represents any amino acid; a bracketed numeral represents the number of or a range of numbers of any amino acid represented by x; a single letter other than x represents a specific amino acid identified by standard single letter amino acid code; and, a bracketed set of two or more single letters represents alternate specific amino acids at a single position;
providing that the ligand does not comprise more than 100 contiguous amino acids of native TACI.

20. The use of a peptide for preparation of a medicament, implantable medical device or implantable medical material, wherein a component of the medicament, device or material binds to CAML, the peptide defined by the amino acid motif:
C-C-x(2)-[FILV]-[ACV]-x(2)-[CS]-x(3)-[KR]-[MR], wherein: x represents any amino acid; a bracketed numeral represents the number of or a range of numbers of any amino acid represented by x; a single letter other than x represents a specific amino acid identified by standard single letter amino acid code; and, a bracketed set of two or more single letters represents alternate specific amino acids at a single position;
providing that the ligand does not comprise more than 100 contiguous amino acids of native TACI.

21. An isolated CAML-binding peptide comprising a sequence of amino acids defined by the motif:
C-C-x(2)-[FILV]-[ACV]-x(2)-[CS)-x(3)-[KR]-[KR], wherein: x represents any amino acid; a bracketed numeral represents the number of or a range of numbers of any amino acid represented by x; a single letter other than x represents a specific amino acid identified by standard single letter amino acid code; and, a bracketed set of two or more single letters represents alternate specific amino acids at a single position;
providing that the ligand does not comprise more than 100 contiguous amino acids of native TACI.

22. The peptide of claim 21, which does not comprise more than 75 contiguous amino acids of native TACI.

23. The peptide of claim 21, which does not comprise more than 50 contiguous amino acids of native TACI.

24. The peptide of claim 21, which does not comprise more than 40 contiguous amino acids of native TACI.

25. The peptide of claim 21, which does not comprise more than 30 contiguous amino acids of native TACI.

26. The peptide of any one of claims 21-25, which does not comprise the amino acid sequence of native E3/6.7K.

27. The peptide of claim 26, which comprises a part of the amino acid sequence of native E3/6.7K.

28. The peptide of any one of claims 21-27, further providing that the peptide does not consist essentially of native synaptotagnin A, B, I or II; adenovirus E3A/7.1K; or, M147R protein of myxoma virus.

29. The peptide of any one of claims 21-28, wherein the motif is:
C-C-x(2)-[ILV]-[ACV]-x(2)-[CS]-x(3)-[KR]-[KR]; wherein x, bracketed numerals, letters and bracketed letters are as defined.

30. The peptide of any one of claims 21-28, wherein the motif is:
C-x(3,5)-C-C-x(2)-[FILV]-[ACV]-x(2)-[CS]-x(3)-[KR]-[KR]; wherein x, bracketed numerals, letters and bracketed letters are as defined.

31. The peptide of any one of claims 21-28, wherein the motif is:
L-x(2,3)-C-x(4,5)-C-C-x(2)-[ILV]-[ACV]-x(2)-[CS]-x(3)-[KR]-[KR]; wherein x, bracketed numerals, letters and bracketed letters are as defined.

32. The peptide of any one of claims 21-28, wherein the motif is:
L-x(1,2)-Cx(5)-C-C-x(2)-[ILV][ACV]-x(2)-[CS]-x(3)-[KR]-[KR]; wherein x, bracketed numerals, letters and bracketed letters are as defined.

33. The peptide of claim 21 consisting essentially of an amino acid sequence selected from the group consisting of: SEQ ID NO: 1-5.

34. The peptide of any one of claims 21-32, comprising an amino acid sequence selected from the group consisting of SEQ ID NO: 1-5.

35. The peptide of any one of claims 21-32, comprising an amino acid sequence selected from the group consisting of SEQ ID NO:6-19.

36. The peptide of any one of claims 21-35, further comprising a detectable label.

37. The peptide of any one of claims 21-35, wherein the peptide is capable of modulating CAML activity.

38. A chimeric peptide comprising a peptide according to any one of claims 21-37, and a heterologous peptide.

39. An isolated nucleic acid encoding a single peptide according to any one of claims 21-38.

40. An isolated nucleic acid encoding a peptide according to any one of claims 21-37, and one or more peptides heterologous to the peptide according to claims 21-37.

41. A vector comprising a nucleic acid according to claim 39 or 40.

42. A cell comprising a nucleic acid according to claim 39 or 40, or a vector according to claim 41.

43. A viral vector comprising a nucleic acid according to claim 39 or 40.

44. A medicament, implantable medical device or implantable medical material comprising a peptide according to any one of claims 21-38, a nucleic acid according to claim 39 or 40, or a vector according to claim 41 or 43.

45. An implantable medical device or material comprising a peptide according to any one of claims 21-38, a nucleic acid according to claim 39 or 40, or a vector according to claim 41 or 43, wherein the device or material is selected from: gauze, membrane, a catheter, an adventitial wrap, an artificial graft, a stent and a shunt.


SEQUENCE LISTING (25 sequences)

SEQ ID NO: 1 - protein, 26 aa, Adenovirus serotype Ad2
LALLCLRVAACCTHVCTYCQLFKRWG

SEQ ID NO: 2 - protein, 26 aa, Adenovirus serotype Ad5
LTLLCLRLAACCVHICIYCQLFKRWG

SEQ ID NO: 3 - protein, 26 aa, Adenovirus serotype Ad3
VLILCYLYTPCCAYLVILCCWFKKWG

SEQ ID NO: 4 - protein, 27 aa, Homo sapiens
VYSTLGLCLCAVLCCFLVAVACFLKKR

SEQ ID NO: 5 - protein, 28 aa, Mus musculus
LYCTLGVCLCAIFCCFLVALASFLRRRG

SEQ ID NO: 6 - protein, 14 aa, Human adenovirus type 3
CCAYLVILCCWFKK

SEQ ID NO: 7 - protein, 14 aa, Discopyge ommata
CCFCICKKCLLKKK

SEQ ID NO: 8 - protein, 14 aa, Discopyge ommata
CCLCICKKCCCKKK

SEQ ID NO: 9 - protein, 14 aa, Bos taurus
CCFCICKKCLFKKK

SEQ ID NO: 10 - protein, 14 aa, Gallus gallus
CCFCLCKKCLFKKK

SEQ ID NO: 11 - protein, 14 aa, Homo sapiens
CCFCICKKCLFKKK

SEQ ID NO: 12 - protein, 14 aa, Mus musculus
CCFCVCKKCLFKKK

SEQ ID NO: 13 - protein, 14 aa, Rattus norvegicus
CCFCVCKKCLFKKK

SEQ ID NO: 14 - protein, 14 aa, Mus musculus
CCFCICKKCCCKKK

SEQ ID NO: 15 - protein, 14 aa, Rattus norvegicus
CCFCICKKCCCKKK

SEQ ID NO: 16 - protein, 14 aa, Homo sapiens
CCFLVAVACFLKKR

SEQ ID NO: 17 - protein, 14 aa, Mus musculus
CCFLVALASFLRRR

SEQ ID NO: 18 - protein, 14 aa, Human adenovirus type 5
CCVHICIYCQLFKR

SEQ ID NO: 19 - protein, 14 aa, Myxoma virus
CCTGLASVCKYTKK

SEQ ID NO: 20 - DNA, 238 bp, Adenovirus Serotype, Ad2 Wild-Type
taagtatatgagcaattcaagtaactctacaagcttgtctaatttttctggaattggggtcggggttatccttactcttgtaattctgtttattcttatactagcacttctgtgccttagggttgccgcctgctgcacgcacgtttgtacctattgtcagctttttaaacgctgggggcaacatccaagatgaggtacatgattttaggcttgctcgcccttgcggcagtctgcagcg

SEQ ID NO: 21 - DNA, 201 bp, Artificial Sequence
  Description of Artificial Sequence  Polymerase Chain Reaction (PCR) Nucleic Acid Product
acaccaccatgagcaattcaagtaactctacaagcttgtctaatttttctggaattggggtcggggttatccttactcttgtaattctgtttattcttatactagcacttctgtgccttagggttgccgcctgctgcacgcacgtttgtacctattgtcagctttttaaacgctgggggcaacatccaagataagggaatt

SEQ ID NO: 22 - DNA, 26 bp, Artificial Sequence
  Description of Artificial Sequence Forward Primer
accaccatgagcaattcaagtaactc

SEQ ID NO: 23 - DNA, 24 bp, Artificial Sequence
  Description of Artificial Sequence Reverse Primer
ccttatcttggatgttgcccccag

SEQ ID NO: 24 - protein, 61 aa, Adenovirus, Ad2 E3/6.7K
MSNSSNSTSLSNFSGIGVGVILTLVILFILILALLCLRVAACCTHVCTYCQLFKRWGQHPR

SEQ ID NO: 25 - protein, 63 aa, Adenovirus, Ad5 E3/6.7K
MNNSSNSTGYSNSGFSRIGVGVILCLVILFILILTLLCLRLAACCVHICTYCQLFKRWGRHPR